(19) United States
(12) Patent Application Publication
(10) Pub. No.: US 2010/0222601 A1
(43) Pub. Date: Sep. 2, 2010

(54) SYNTHESIS OF CYCLOPENTADIENE DERIVATIVES

(75) Inventors: Ouardia Tahiri, Frankfurt (DE); Robert L. Jones, Oakland, CA (US); Michael Schiendorfer, Frankfurt (DE); Reynald Chevalier, Frankfurt (DE)

Correspondence Address: BASELL USA INC., NEWTOWN SQUARE CENTER, 3801 WEST CHESTER PIKE, BLDG. B, NEWTOWN SQUARE, PA, 19073, US

(73) Assignee: Basell Polyolefine GmbH, Wesseling (DE)

(21) Appl. No.: 12/448,152

(22) PCT Filed: Nov. 21, 2007
(86) PCT No.: PCT/EP2007/062648

§ 371 Date: Jul. 13, 2009

Related U.S. Application Data
(60) Provisional application No. 60/874,562, filed on Dec. 13, 2006.

(30) Foreign Application Priority Data
Dec. 12, 2006 (EP) 06125923.0

(51) Int. Cl. C07D 333/78

(52) U.S. Cl. 549/43

(57) ABSTRACT

A process for preparing cyclopentadiene derivatives having formula (I)

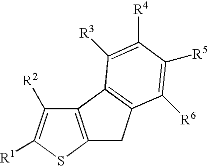

Wherein R¹, R², R³, R⁴, R⁵ and R⁶ equal to or different from each other, are hydrogen atoms or hydrocarbon groups comprising the steps of reacting a substituted or unsubstituted indanon with a base and then with a substituted or unsubstituted haloaceton and treating the obtained product with the Lawesson's reagent.

This application is the U.S. national phase of International Application PCT/EP2007/062648, filed Nov. 21, 2007, claiming priority to European Application 06125923.0 filed Dec. 12, 2006 and the benefit under 35 U.S.C. 119(e) of U.S. Provisional Application No. 60/874,562, filed Dec. 13, 2006; the disclosures of International Application PCT/EP2007/062648, European Application 06125923.0 and U.S. Provisional Application No. 60/874,562, each as filed, are incorporated herein by reference.

The present invention relates to a process for the preparation of cyclopentadiene derivatives of formula (I)

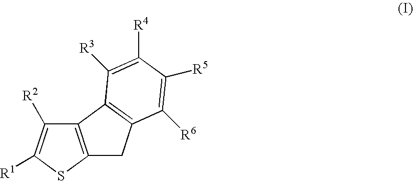

These compounds are fit for the preparation of metallocene complexes useful as catalysts for the polymerization of olefins.

Examples of these cyclopentadiene derivatives are known in the art. In the Ninth International Business Forum on Specialty Polyolefins (SPO 99 Oct. 12-13, 1999, Huston Tex.) metallocene complexes containing a cyclopentadienyl moiety above defined have been presented. These moieties have been synthesized using as starting point thiofene derivatives. The process includes the direct addition of methacrylic acid catalyzed by polyphosphoric acid. To obtain the correspondent Ketone that is then reduced. Even if the yields of this process are quite hight there are several steps to carry out, and then the yield can be further improved. WO02/092564 describes the synthesis of the moiety described above by using a process that involves the formation of a ketone as intermediate. Also in this case the process involve several step and the yield can be improved.

Thus an object of the present invention is to find a process for the synthesis of the compounds having the structure reported above in an easy way in high yields.

The present invention relates to a process for preparing cyclopentadiene derivatives having formula (I)

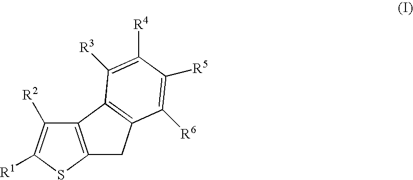

wherein

R¹, R², R³, R⁴, R⁵ and R⁶ equal to or different from each other, are hydrogen atoms or hydrocarbon groups containing from 1 to 40 carbon atoms optionally containing O, S, N, P or Si atoms or they can form a C₄-C₇ ring that can bear substituents; preferably R¹, R², R³, R⁴, R⁵ and R⁶ are hydrogen atoms or linear or branched, cyclic or acyclic, C₁-C₄₀-alkyl, C₂-C₄₀ alkenyl, C₂-C₄₀ alkynyl, C₆-C₄₀-aryl, C₇-C₄₀-alkylaryl or C₇-C₄₀-arylalkyl radicals; optionally containing heteroatoms belonging to groups 13-17 of the Periodic Table of the Elements; more preferably R¹, R², R³, R⁴, R⁵ and R⁶ are hydrogen atoms or C₁-C₂₀-alkyl radicals;

said process comprises the following steps:

a) reacting a compound of formula (II)

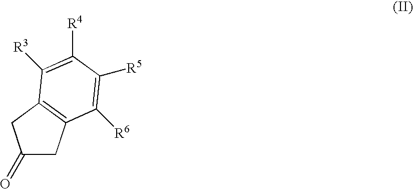

with a base selected from the group consisting of metallic sodium and potassium, sodium and potassium hydroxide and an organolithium compound, wherein the molar ratio between the compound of the formula (II) and said base is at least 1:1; preferably the organolithium compound has formula LiR^{a} wherein R^{a} is a C₁-C₄₀ hydrocarbon group, preferably R^{a} is a C₁-C₄₀-alkyl, C₆-C₄₀-aryl, C₇-C₄₀-alkylaryl or C₇-C₄₀-arylalkyl radical; more preferably R^{a} is a C₁-C₂₀-alkyl or a C₆-C₂₀-aryl radical;

R³, R⁴, R⁵ and R⁶ have been described above; preferably sodium or potassium hydride are used;

b) reacting the reaction product of step a) with a compound of formula (III

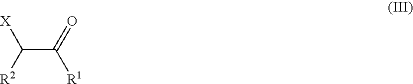

### Wherein:

X is an halogen atom; preferably X is chlorine or bromine; R¹ and R² have been defined above; to obtain a compound of formula (IV)

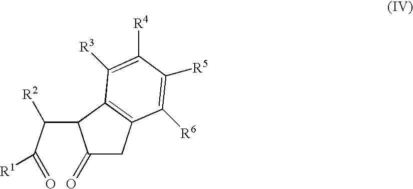

c) treating the compound of formula (IV) obtained in step a) with a compound of formula (V)

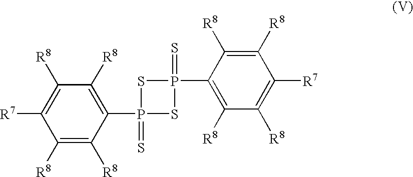

to obtain the compound of formula (I);

wherein R⁷ equal to or different from each other is an hydrogen atom or a hydrocarbon group containing from 1 to 40 carbon atoms and optionally containing O, S, N, P or Si atoms; preferably R⁷ is a OR⁹ group wherein R⁹ is a C₁-C₄₀-alkyl, C₆-C₄₀-aryl, C₇-C₄₀-alkylaryl or C₇-C₄₀-arylalkyl radical; more preferably R⁹ is a C₁-C₂₀-alkyl radical; more preferably R⁹ is a methyl or ethyl radical; R⁸ equal to or different from each other are hydrogen atoms or hydrocarbon group containing from 1 to 40 carbon atoms and optionally containing O, S, N, P or Si atoms; preferably R⁸ are hydrogen atoms; more preferably the compound of formula (V) is 2,4-bis(4-methoxyphenyl)-1,3,2,4-dithiadiphosphetane 2,4-disulfide.

Steps a) and b) The above processes are preferably carried out in an aprotic solvent, either polar or apolar. Said aprotic solvent is preferably an aromatic or aliphatic hydrocarbon, optionally halogenated, or an ether; more preferably it is selected from benzene, toluene, pentane, hexane, heptane, cyclohexane, dichloromethane, diethylether, tetrahydrofurane and mixtures thereof. The above processes are carried out at a temperature ranging from −100° C. to +80° C., more preferably from −80° C. to +70° C. Steps a) and b) can be also carried out “one pot”, i.e. without the isolation of the product obtained in step a). In this case after step a) it can be necessary to vary the temperature conditions before the addition of the compound of formula (III).

The compound of formula (IV) obtained by step b) can be purified by using means well known to the skilled in the art such as crystallization or it can be used directly without the need of a purification step.

Step c) is carried out in an aprotic solvent, either polar or apolar. Preferably it is carried out in an apolar solvent such as an aromatic or aliphatic hydrocarbon; more preferably it is selected from benzene, toluene, pentane, hexane, heptane, cyclohexane and mixtures thereof. The above processes are carried out at a temperature ranging from 25° C. to 200° C., more preferably from 40° C.+150° C., even more preferably from 50° C. to 120° C.

The compound of formula (V) is the Lawesson's reagent reference on the use of this reagent can be found on Tetrahedron 35, 2433 (1979) and Tetrahedron 41, 5061 (1985).

The compounds of formula (I) can be used to synthesize metallocene compounds of formula (VI)

(A-L-A′)MX₂ (VI)

Wherein A is the cyclopentadienyl radical of a compound of formula (I); L is a bridge connecting A and A′; A′ is a group containing a cyclopentadienyl radical; M is a metal of groups 3-6 of the periodic table; preferably of group 4, and X is an halogen atom or an hydrocarbon radical containing from 1 to 40 carbon atoms, optionally containing heteroatoms of groups 14-16 of the periodic table.

### EXAMPLES

### General Procedures

All operations were performed under nitrogen by using conventional Schlenk-line techniques. Solvents were purified by degassing with N₂ and passing over activated (8 hours, N₂ purge, 300° C.) Al₂O₃, and stored under nitrogen. n-BuLi (Aldrich) was used as received.

### Example 1

### Steps a) and b)

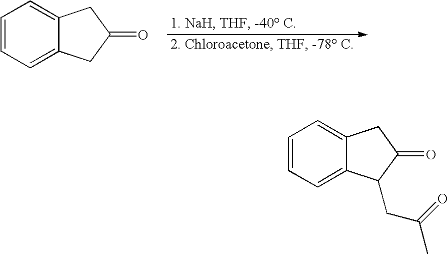

To a suspension of sodium hydride (2.19 g, 86.69 mmol) in THF is dropwise added at −40° C. a solution of 2-indanone (10.79 g, 80.01 mmol) in THF. After complete addition, the obtained reaction mixture is allowed to warm up to room temperature (rt) and stirred 4 additional hours at rt. Then the previous reaction mixture is treated at −78° C. with a solution of chloroacetone (7.79 g, 81.67 mmol) in THF. The obtained green suspension is slowly warmed up to rt over a night. 250 ml of water are slowly added and the layers are separated; the aqueous one is extracted twice with ether; the organic layers are all collected, dried over magnesium sulfate and all solvents are removed to live 15.97 g (66% GC-purity, 70% yield) red brown oil which is directly used without further treatment.

### Step c)

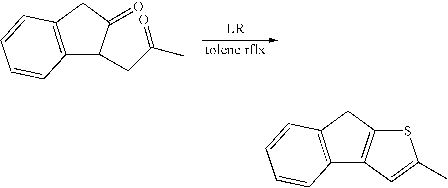

A mixture of 1,4-diketone obtained in step b) (15.97 g, 66% purity, 56 mmol) and 2,4-bis(4-methoxyphenyl)-1,3,2,4-dithiadiphosphetane 2,4-disulfide (Lawesson's reagent, LR) (27.21 g, 65.26 mmol) are refluxed in Toluene for 2 h30. Then the obtained mixture is concentrated to the half volume and is filtered through a celite and the obtained filtrate is concentrated in vacuum to live a dark brown oil which is purified by flash chromatography to yield 7.56 g of pure final product (yield 72.6%). ¹H NMR (CD₂Cl₂): δ=2.53 (s, 3H, Me), 3.76 (s, 2H, CH₂-central ring), 6.95 (s, 1H, 3-H), 7.12 (t, 1H, 5-H), 7.25 (t, 1H, 6-H), 7.43 (d, 2H, 4-H and 7-H) ppm.

### Examples 2-7

Examples 2-7 have been carried out by following the procedure of example 1 and by changing the compounds of formula (II) and (III). The results are reported on table 1

TABLE 1

| | | | | Overall |
|---|---|---|---|---|
| | Compound of | Compound of | | yield |
| Ex | formula (II) | Formula (III) | Compound of formula (I) | % |
| 2 | 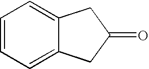 | 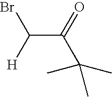 | 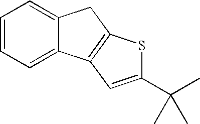 | 75 |
| 3a | 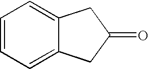 | 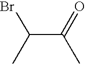 | 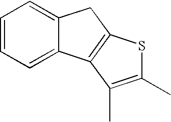 | 60 |
| 3b | 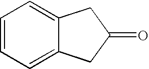 | 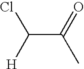 | 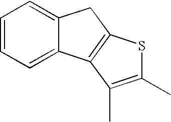 | 84 |
| 4 | 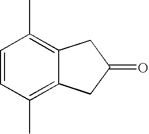 | 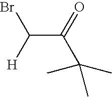 | 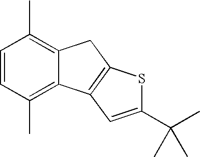 | 48 |
| 5 | 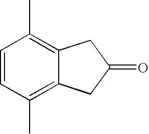 | 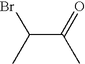 | 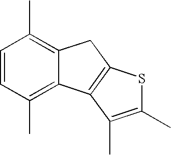 | 45 |
| 6 | 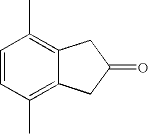 | 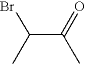 | 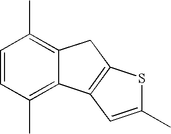 | 48 |
| 7 | 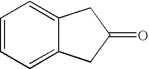 | 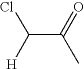 | 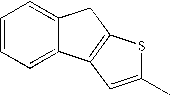 | 64 |

1. A process for preparing cyclopentadiene derivatives having formula (I): 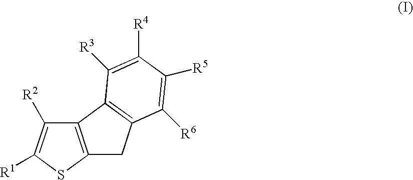
wherein
R¹, R², R³, R⁴, R⁵ and R⁶ equal to or different from each other, are hydrogen atoms or hydrocarbon groups containing from 1 to 40 carbon atoms optionally containing O, S, N, P or Si atoms, or they form a C₄-C₇ ring that bears C₁-C₂₀ hydrocarbon substituents,
said process comprising the following steps:
a) reacting a compound of formula (II): 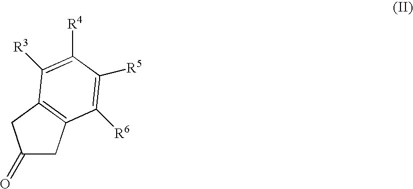
with a base selected from the group consisting of metallic sodium and potassium, sodium and potassium hydroxide and an organolithium compound, wherein the molar ratio between the compound of the formula (II) and said base is at least 1:1;
b) reacting the reaction product of step a) with a compound of formula (III): 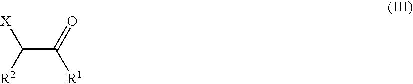
wherein
X is an halogen atom,
to obtain a compound of formula (IV): 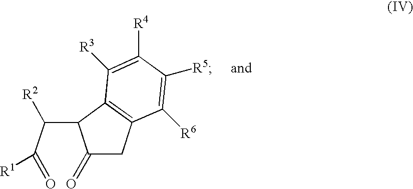
c) treating the compound of formula (IV) obtained in step b) with a compound of formula (V): 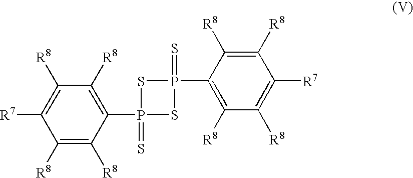
to obtain the compound of formula (I),
wherein R⁷ equal to or different from each other is an hydrogen atom or hydrocarbon group containing from 1 to 40 carbon atoms and optionally containing O, S, N, P or Si atoms; and
R⁸ equal to or different from each other are hydrogen atoms or a hydrocarbon group containing from 1 to 40 carbon atoms and optionally containing O, S, N, P or Si atoms.

2. The process according to claim 1, wherein the base used in step a) is sodium or potassium hydride.

3. The process according to claim 1, wherein in the compound of formula (V), R⁷ is an OR⁹ group, wherein R⁹ is a C₁-C₄₀-alkyl, C₆-C₄₀-aryl, C₇-C₄₀-alkylaryl or C₇-C₄₀-arylalkyl radical.

4. The process according to claim 1 wherein steps a) and b) are carried out without the isolation of the product obtained in step a).

5. The process according to claim 1 wherein the product of formula (IV) obtained in step b) is used without a purification step.

6. The process according to claim 1 wherein steps a) and b) are carried out at a temperature ranging from −100° C. to +80° C.

7. The process according to claim 1 wherein steps a) and b) are carried out at a temperature ranging from 25° C. to +200° C.
